(19) United States
(12) Patent Application Publication
(10) Pub. No.: US 2021/0048443 A1
(43) Pub. Date: Feb. 18, 2021

(54) METHOD FOR DIAGNOSING HAEMOSTASIS DISORDERS USING ACTIVATED CHARCOAL

(71) Applicant: UNIVERSITÉ DE NAMUR ASBL, Namur (BE)

(72) Inventors: Jonathan Douxfils, Courriére (BE); Damien Claude Joseph Gheldof, Floriffoux (BE); Jean-Michel Paul Nicolas Dogné, Grivegnée (BE)

(21) Appl. No.: 16/967,468

(22) PCT Filed: Feb. 6, 2019
(86) PCT No.: PCT/EP2019/052903

§ 371 Date: Aug. 5, 2020

(30) Foreign Application Priority Data
Feb. 6, 2018 (EP) 18155295.1

(51) Int. Cl. G01N 33/86; C12Q 1/56

(52) U.S. Cl. CPC G01N 33/86; G01N 2800/224; C12Q 1/56

(57) ABSTRACT

The application provides a method for the in vitro diagnosis of a haemostasis disorder in a plasma sample obtained from a subject comprising the steps of a) contacting a plasma sample obtained from said subject with activated charcoal; b) recovering said plasma sample from said activated charcoal; and c) determining the coagulation ability of said plasma sample obtained under step (b) wherein the ability of said plasma sample to coagulate is indicative of the presence, progression, or severity of a haemostasis disorder in said subject, and optionally of the nature of the haemostasis disorder.

### TECHNICAL FIELD

The present invention is situated in the field of methods for diagnosing haemostasis disorders. More particularly, the invention provides methods and kits for diagnosing haemostasis disorders, such as in patients treated with an anticoagulant.

### BACKGROUND OF THE INVENTION

Overall, patients with unprovoked venous thromboembolism (VTE) have a high risk of recurrence once off anticoagulant therapy. Indeed, the cumulative risk for recurrent VTE is approximately 10% at 1 year, 30% at 5 years, and 50% at 10 years. Routine and specific clotting tests are useful screening tests to detect a haemostasis disorder. Several inherited conditions (e.g. activated protein C resistance, protein C and S deficiency, antithrombin deficiency and elevated factor VIII) increase the risk for recurrent thromboembolism. The degree of risk depends on the previous condition and therefore, tests for thrombophilic risk factors should not be performed as general population screening but rather in selected patients who had a thromboembolic event or who have known family history. These patients are often treated for their thromboembolic disease and therefore, the interpretation of the result is complicated by the effect of anticoagulation medications. Furthermore, patients taking antithrombotic therapy may require coagulation testing to diagnose the haemostasis disorder that has developed after the initiation of the treatment, such as vitamin K deficiency, liver disease, or acquired haemophilia A. The National Institute for Health and Care Excellence (NICE) guideline on venous thromboembolic diseases recognizes the clinical relevance of these tests, though, their use is not comfortable and optimal since it implies that the patient has to be unprotected/untreated during the investigation phase to allow a reliable diagnosis.

Since 2008, a novel class of anticoagulant agents reached the market of anticoagulation: the direct oral anti-coagulants (DOACs). These compounds include one thrombin inhibitor (dabigatran etexilate—Pradaxa®) and three factor Xa inhibitors (rivaroxaban—Xarelto®; apixaban—Eliquis®; and edoxaban—Lixiana®) and do not require routine monitoring of anticoagulation, in contrast to previously used vitamin K antagonists and low molecular weight heparins (LMWHs). However, evidence suggests that point measurement may be useful in several situations and that specific tests should be employed. On the other hand, the introduction of drugs targeting directly one factor of the coagulation will affect several haemostasis test that involve the concerned clotting factor, leading to false-positive or false-negative results. Thus, there is a need to avoid the effect of direct anticoagulants in coagulation testing to facilitate the diagnosis and to ensure a proper assessment of the aetiology of a thrombotic event.

Tools have been identified for the removal of low molecular weight compounds such as dyes from a blood sample (such as in WO9934914A1). However, these are typically envisaged for use on large batches of plasma. Moreover the relevance of these techniques in the context of diagnosing a haemostasis disorder has not yet been considered.

### SUMMARY OF THE INVENTION

The present inventors have found that activated charcoal can be used in the preparation of plasma for in vitro tests for diagnosing a haemostasis disorder to remove direct anticoagulants (e.g. DOACs) from a plasma sample. The method as disclosed herein allows determining the coagulation ability of plasma obtained from a subject, in absence of the interfering effect of direct anticoagulants (e.g. DOACs) on the coagulation ability, thereby allowing accurate detection of the presence of a haemostasis disorder in said subject. The invention moreover allows the determination of a risk of haemostasis disorder based on the presence of anticoagulants in a sample.

In particular embodiments, the method for the in vitro diagnosis or of a haemostasis disorder in a plasma sample obtained from a subject comprises the steps of contacting a plasma sample obtained from said subject with activated charcoal so as to allow adsorption of said DOAC onto said charcoal; separating said adsorbed activated charcoal from the sample, and determining the coagulation ability of said plasma sample so obtained. In particular embodiments, the method comprises (a) contacting a plasma sample obtained from said subject with activated charcoal; (b) recovering said plasma sample from said activated charcoal; and c) determining the coagulation ability of said plasma sample obtained under step (b). In these methods, the ability of said plasma sample to coagulate is effectively indicative of the presence, progression, or severity of a haemostasis disorder in said subject, and optionally of the nature of the haemostasis disorder. Accordingly, in particular embodiments, said method comprises step (d) which encompasses, determining, based on said coagulation ability of said plasma, the presence, progression, or severity of a haemostasis disorder in said subject.

In particular embodiments, the plasma sample is recovered from the activated charcoal by passing the plasma sample through a filter. In further embodiments, the filter is a filter with a pore size comprised between 0.22 and 0.65 micrometer, such that platelets, platelet fragment and/or blood cells which were still present in the plasma, as well as the activated charcoal, are removed from the plasma. Indeed, in particular embodiments, the methods are characterized in that the platelets and DOACs are removed from the sample in one step, and do not comprise a separate step of removing the platelets from said plasma sample.

The method as disclosed herein allows a fast and reliable assessment of a haemostasis disorder by in vitro diagnostic assays, for instance, for determining whether observed decreased coagulation ability (e.g. lack of blood clotting) can be attributed to a haemostasis disorder or to a decreased coagulation ability (e.g. lack of blood clotting) due to the presence of anticoagulating agents in the sample of the patient. In particular embodiments, the step of determining, based on said coagulation ability of said plasma, the presence, progression, or severity of a haemostasis disorder in said subject is performed by comparison of said coagulation ability of said sample with a standard or reference value.

Accordingly, a first aspect provides a method for the in vitro diagnosis of a haemostasis disorder in a plasma sample obtained from a subject comprising the steps of

a) contacting a plasma sample obtained from said subject with activated charcoal;
b) recovering said plasma sample from said activated charcoal; and
c) determining the coagulation ability of said plasma sample obtained under step (b);
wherein the ability of said plasma sample to coagulate is indicative of the presence, progression, or severity of a haemostasis disorder in said subject, and optionally of the nature of the haemostasis disorder. In particular embodiments, said step (b) is performed by passing the plasma sample through a filter. In further particular embodiments, said plasma sample has a pore size from 0.22 to 0.65 μm. In particular embodiments, the step of recovering the plasma from the activated charcoal comprises a centrifugation step. In particular embodiments, the activated charcoal has a concentration of at least 3 mg/ml, preferably at least 5 mg/ml. In particular embodiments, the plasma sample is contacted with activated charcoal for at least 2 minutes, preferably at least 5 minutes.

In particular embodiments, the method for the in vitro diagnosis of a haemostasis disorder as disclosed herein does not comprise prior to step (c) an additional step of removing one or more of platelets, platelet fragments and residual blood cells from the plasma sample.

In particular embodiments, the step of determining the coagulation ability of said plasma sample obtained under step (b) is performed by contacting the plasma sample with a coagulation activator.

In particular embodiments, the coagulation activator is selected from the group consisting of human calcium thrombin, rabbit or recombinant human tissue factor, synthetic phospholipids, Russel's viper venom, ecarin, textarin or silica, colloidal silica activator, thrombomodulin, activated protein C, lyophilized bovine thrombin and chromogenic substrate of thrombin CBS 61.50, factor V activator from snake venom and factor Va-dependent prothrombin activator isolated from snake venom.

In particular embodiments, the step of determining the coagulation ability of said plasma sample obtained under step (b) further comprises contacting said plasma sample with an immune depleted serum or plasma prior to step (c).

In particular embodiments, the immune depleted serum or plasma is selected from the group consisting of Factor VIII or IX or X or XI or XII or XIII or VII or V or II deficient serum or plasma.

In particular embodiments, the step of determining the coagulation ability of said plasma sample obtained under step (b) is performed by a coagulation test chosen from the list comprising prothrombin time (PT), activated thromboplastin time (aPTT), lupus anticoagulant test, fibrinogen assays (both Clauss and PT derived-fibrinogen methods), thrombin time, coagulation factor activity assays (FVIII, FIX, X, XI, XII, XIII; VII, V, II, X), activated protein C resistance (APCR) assay, Protein C activity assay, Protein S activity assay, antithrombin activity assay and thrombin generation assay.

In particular embodiments, the step of determining the coagulation ability of said plasma sample obtained under step (b) is determined using a blood clotting-based method for determining Fibrinogen deficiency, Prothrombin deficiency, Factor V deficiency, Factor V Leiden, Protein C deficiency, protein S deficiency, antiplasmin deficiency, antithrombin deficiency, plasminogen deficiency, Elevated D-Dimer, antiphospholipid syndrome, heparin induced thrombocytopenia, Combined Factor V and VIII deficiency, Factor VII deficiency, Factor VIII deficiency (Haemophilia A), Factor IX deficiency (Haemophilia B), Factor X deficiency, Factor XI deficiency, Factor XIII deficiency, Glanzmann's thrombasthenia, Bernard Soulier Syndrome, Wiskott-Aldrich Syndrome or Leukocyte Adhesion deficiency, and further provides an indication of the nature of said haemostasis disorder.

In particular embodiments, the subject is a patient which has been treated with a direct anticoagulant, preferably a direct oral anticoagulant (DOAC).

In particular embodiments, the subject is a subject of which the medical history is not known and/or cannot be ascertained.

A further aspect provides a diagnostic kit for the in vitro diagnosis of a haemostasis disorder comprising
activated charcoal; and
one or more compounds required for the in vitro diagnosis of a haemostasis disorder. In particular embodiments, the kit comprises a vial comprising a filter. In further embodiments, the filter has a pore size from 0.22 to 0.65 μm, such as a pore size of 0.45 μm. The invention further provides kits for preparing a sample for diagnostic testing, more particularly of a haemostasis disorder, said kits comprising a vial of a volume between 100 μl and 10000 μl; activated charcoal and a filter with a pore size of from 0.22 to 0.65 μm.

In a further aspect the invention comprises a method for preparing a sample for in vitro diagnosis of a haemostasis disorder comprising the steps of

a) contacting a volume of between 100 μl and 10000 μl of said plasma sample with activated charcoal in a vial; and
b) recovering said plasma sample from said activated charcoal by passing said plasma sample through a filter with a pore size from 0.22 to 0.65 μm.

### BRIEF DESCRIPTION OF THE FIGURES

FIG. 1. illustrates an exemplary standard preparation method for obtaining platelet-poor plasma by centrifugation.

FIG. 2. illustrates the activated Partial Thromboplastin Time (aPTT) (a) and Prothombin Time (PT) (b) of platelet-poor plasma with increasing concentrations of direct oral anti-coagulants (DOACs).

FIG. 3. illustrates an exemplary spin filter loaded with activated charcoal as disclosed herein (2) which is placed into a sealable Eppendorf tube (3). Blood plasma (1) may be loaded into the spin filter and upon centrifugation of the spin filter, the plasma, which is free of DOACS, platelets and activated charcoal, may be recovered in the sealable Eppendorf tube (3).

FIG. 4. illustrates the aPTT (a) and PT (b) of blood plasma subjected to the method as disclosed herein with increasing concentrations of DOACs.

FIG. 5. illustrates the aPTT (a) and PT (b) of blood plasma subjected to the method as disclosed herein with increasing concentrations of Rivaroxaban and increasing concentrations of activated charcoal.

FIG. 6. illustrates the clotting times obtained by Partial Thromboplastin Time-Lupus anticoagulant Screen (PTT LA), PTT LA confirm (Staclot LA), Dilute Russel Viper Venom Time screen (DRVVT) and DRVVT confirm of a plasma sample of patient suspected to suffer from LA, which was either platelet-poor plasma (checked pattern) or blood plasma subjected to the method as disclosed herein (grey).

FIG. 7: DOAC impact on LA-diagnosis first line assays; (A) DRVVT Screen; (B) DRVVT confirm; (C) PTT-LA

### DETAILED DESCRIPTION OF THE INVENTION

Before the present method and devices used in the invention are described, it is to be understood that this invention is not limited to particular methods, molecules, or uses described, as such methods, molecules, or uses may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The terms “comprising”, “comprises” and “comprised of”, as used herein, are synonymous with “including”, “includes” or “containing”, “contains”, and are inclusive or open-ended and do not exclude additional, non-recited members, elements or method steps.

The terms “comprising”, “comprises” and “comprised of” also include the term “consisting of”.

The term “about”, as used herein, when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination. The impact of direct anticoagulants (e.g. DOACs) on screening tests such as the prothrombin time (PT) or the activated thromboplastin time (aPTT) is problematic for laboratories which are not always aware of the therapy being taken by the patient. Rapid and reliable determination of the root cause of a life threatening bleeding in unconscious or trauma patients is needed to ensure adequate patient management. The availability of screening tests that can be either sensitive or insensitive to direct anticoagulants (e.g. DOACs) may rapidly inform the laboratory on the presence of direct anticoagulants (e.g. DOACs). This will spare useless, expensive and time-consuming investigations and will provide reliable diagnosis. In addition, the presence of direct anticoagulants (e.g. DOACs) in a sample affects tests used to assess haemostasis disorders that involve the targeted clotting factor, leading to false-positive or false-negative results. While some of these tests are adapted to be insensitive to heparins, they are not designed to be performed in the presence of direct anticoagulants (e.g. DOACs). Thus, there is a need to avoid the effect of direct anticoagulants (e.g. DOACs) in these coagulation tests to provide a reliable assessment of hypercoagulability.

The present inventors have found that activated charcoal can be used in the preparation of plasma for in vitro tests for diagnosing a haemostasis disorder to remove direct anticoagulants (e.g. DOACs) from a plasma sample. The method as disclosed herein allows determining the coagulation ability of plasma, in absence of the interfering effect of direct anticoagulants (e.g. DOACs) on the coagulation ability. Furthermore, when the plasma sample is, in addition to the treatment with activated charcoal, filtered by passing the plasma sample through a filter, for example a filter with pores sizes comprised between 0.22 and 0.65 micrometer, or more particularly between 0.22 and 0.65 micrometer, such as between 0.40 and 0.65 micrometer, platelets, platelet fragment and/or blood cells which were still present in the plasma, as well as the activated charcoal, can be removed from the plasma.

The method as disclosed herein allows a fast and reliable assessment of a haemostasis disorder by in vitro diagnostic assays, for instance, for determining whether observed decreased coagulation ability (e.g. lack of blood clotting) can be attributed to a haemostasis disorder or to a decreased coagulation ability (e.g. lack of blood clotting) due to the presence of anticoagulating agents in the sample of the patient. By using activated charcoal in combination with a filter, for example a filter with pores sizes comprised between 0.22 and 0.65 micrometer, such as a filter of 0.45 μm as provided by the method as disclosed herein, valuable time can be saved during the assessment by reducing the number of steps and/or centrifugation time required to obtain a plasma sample (substantially) free of direct anticoagulants, platelets, platelet fragment and/or blood cells which can be used reliably to assess a blood coagulation disorder.

The term haemostasis disorder as used herein refers to a disorder in the equilibrium between bleeding and clotting. The disorder may be either congenital or acquired. The haemostasis disorder can generate a risk excessive bleeding or thrombosis.

The methods of the invention also allow the

Accordingly, a first aspect provides a method for the in vitro diagnosis of a haemostasis disorder in a subject comprising the steps of

a) contacting a plasma sample obtained from said subject with activated charcoal;
b) recovering said plasma sample from said activated charcoal; and
c) determining the coagulation ability of said plasma sample obtained under (b);
wherein the ability of said plasma sample to coagulate is indicative of the presence progression or severity of a haemostasis disorder in said subject, and optionally of the nature of the haemostasis disorder.

Well-regulated haemostasis is crucial for health and both inadequate coagulation, such as in the inherited disorder hemophilia, and excessive coagulation, as occurs in thrombophilia, can have drastic consequences such as hemorrhage and thrombosis. Haemostasis disorders can be separated into two main groups i.e. inherited or acquired and are further classified in coagulation factor deficiencies, platelet disorders, vascular disorders and fibrinolytic defects. Non-limiting examples of haemostasis disorders are Fibrinogen deficiency, Prothrombin deficiency, Factor V deficiency, Factor V Leiden, Protein C deficiency, protein S deficiency, antiplasmin deficiency, antithrombin deficiency, plasminogen deficiency, Elevated D-Dimer, antiphospholipid syndrome, heparin induced thrombocytopenia, Combined Factor V and VIII deficiency, Factor VII deficiency, Factor VIII deficiency (Haemophilia A), Factor IX deficiency (Haemophilia B), Factor X deficiency, Factor XI deficiency, Factor XIII deficiency, Glanzmann's thrombasthenia, Bernard Soulier Syndrome, Wiskott-Aldrich Syndrome and Leukocyte Adhesion deficiency). Haemostasis disorders may be diagnosed by a variety of methods for measuring the coagulation ability of the plasma, including but not limited to a chromogenic anti-factor Xa activity assay, activated partial thromboplastin time assay, prothrombin time, thrombin time, activated clotting time, thromboelastography, thrombin generation assay, reptilase time, dilute Russell's viper venom time, ecarin clotting time, kaolin clotting time, International Normalized Ratio (INR), fibrinogen testing (Clauss), thrombin time (TT), mixing time, and euglobulin lysis time. These methods help to determine various coagulation parameters, and are known to the skilled person. Haemostasis disorders that lead to overactive clotting are mostly treated with a coagulation inhibitor. The coagulation inhibitor (also referred to herein as anticoagulant) is a molecule that inhibits coagulation process. Exemplary coagulation inhibitors include, but are not limited to, antithrombin activators (e.g., unfractionated heparin and LMWH), factor IIa inhibitors, and factor Xa inhibitors. An anticoagulant effect is any effect of a coagulation inhibitor that results from its blockage of the propagation of the coagulation cascades. Non-limiting examples of anticoagulation effects include upregulation of antithrombin activity, decreased Factor Xa activity, decreased Factor IIa activity, increased blood loss, and any other conditions wherein the activity or concentrations of clotting factors are altered in such a way as to inhibit blood clot formation.

As indicated above, haemostasis disorders typically increase the risk of a subject for diseases and disorders such as (excessive) bleeding or hemorrhagic diathesis), disseminated intravascular coagulopathy, thrombosis etc. Accordingly, the methods of the invention allow the determination of an increased risk of diseases or disorders resulting from a haemostasis disorder. The term “direct anticoagulant” as used herein refers to anticoagulants that directly target the enzymatic activity of thrombin and/or factor Xa. Direct anticoagulants include oral and parental direct thrombin (Factor IIa) inhibitors and oral direct factor Xa inhibitors. Non-limiting examples of direct anticoagulants include anticoagulants such as dabigatran etexilate (PRADAXA®), rivaroxaban (XARELTO®), apixaban (ELIQUIS®), edoxaban (LIXIANA®), fondaparinux (ARIXTRA®), and argatroban (ARGATROBAN®).

The chemical name for oral anticoagulant PRADAXA®, dabigatran etexilate mesylate, a direct thrombin inhibitor, is β-Alanine, N-[[2-[[[4-[[[(hexyloxy)carbonyl]amino]imino-methyl]phenyl]amino]methyl]-1-methyl-1H-benzimidazol-5-yl]carbonyl]-N-2-pyridinyl-, ethylester, methanesulfonate. Dabigatran and its acyl glucuronides are competitive, direct thrombin inhibitors. Because thrombin (Factor IIa, serine protease) enables the conversion of fibrinogen into fibrin during the coagulation cascade, its inhibition prevents the development of a thrombus.

Rivaroxaban, a factor Xa inhibitor, is the active ingredient in XARELTO®, and has the chemical name 5-Chloro-N-({(5S)-2-oxo-3-[4-(3-oxo-4-morpholinyl)phenyl]-1,3-oxazolidin-5-yl}methyl)-2-thiophenecarboxamide. Rivaroxaban is a pure (S)-enantiomer. XARELTO® is an orally bioavailable factor Xa inhibitor that selectively blocks the active site of factor Xa and does not require a cofactor (such as Anti-thrombin III) for activity.

Apixaban or ELIQUIS® is 1-(4-methoxyphenyl)-7-oxo-6-[4-(2-oxopiperidin-1-yl)phenyl]-4,5-dihydropyrazolo[3,4-c]pyridine-3-carboxamide. It is an orally administered direct factor Xa inhibitor approved in Europe and presently undergoing phase III trials in the U.S. for the prevention of venous thromboembolism etc.

Edoxaban or LIXIANA® is N′-(5-chloropyridin-2-yl)-N-[(1S,2R,4S)-4-(dimethylcarbamoyl)-2-[(5-methyl-6,7-dihydro-4H-[1,3]thiazolo[5,4-c]pyridine-2-carbonyl)amino]cyclohexyl]oxamide. Edoxaban is a direct factor Xa inhibitor, and it has been approved in Japan for use in preventing venous thromboembolism.

ARIXTRA® is fondaparinux sodium. It is a synthetic and specific inhibitor of activated Factor X (Xa). Fondaparinux sodium is methyl O-2-deoxy-6-0-sulfo-2-(sulfoamino)-a-D-glucopyranosyl-(1→4)-0-P-D-glucopyra-nuronosyl-(1→4)-0-2-deoxy-3,6-di-0-sulfo-2-(sulfoamino)-a-D-glucopyranosyl-(1→4)-0-2-0-sulfo-a-L-idopyranuronosyl-(1→4)-2-deoxy-6-0-sulfo-2-(sulfoamino)-a-D-glucopyranoside, decasodium salt. Neutralization of Factor Xa interrupts the blood coagulation cascade and thus inhibits thrombin formation and thrombus development. Only fondaparinux can be used to calibrate the anti-Xa assay. The international standards of heparin or LMWH are not appropriate for this use.

ARGATROBAN® is a synthetic direct thrombin (Factor IIa) inhibitor, derived from L-arginine. The chemical name for ARGATROBAN® is 1-[5-[(aminoiminomethyl) amino]-1-oxo-2-[[(1,2,3,4-tetrahydro-3-methyl-8-quinolinyl)sulfonyl] amino]pentyl]-4-methyl-2-piperidinecarboxylic acid, monohydrate. ARGATROBAN® is a direct thrombin inhibitor that reversibly binds to the thrombin active site. ARGATROBAN® does not require the co-factor antithrombin III for antithrombotic activity. As used in the present invention, the term “plasma sample” refers to a sample obtained from a subject or patient to be diagnosed with the method as disclosed herein.

The term “plasma” as used herein is as conventionally defined and comprises fresh plasma, thawed frozen plasma, solvent/detergent-treated plasma, processed plasma, or a mixture of any two or more thereof. Preferably, plasma is fresh plasma. Plasma is usually obtained from a sample of whole blood, provided or contacted with an anticoagulant, (e.g., heparin, citrate, oxalate or EDTA). Subsequently, cellular components of the blood sample are separated from the liquid component (plasma) by an appropriate technique, typically by centrifugation (e.g. 15 min at 1500 g at room temperature to separate the plasma from the red blood cells). The term “plasma” refers to a composition which does not form part of a human or animal body. The term “plasma” may in certain embodiments specifically include processed plasma, i.e., plasma subjected after its separation from whole blood to one or more processing steps which alter its composition, specifically its chemical, biochemical, or cellular composition.

The term “platelet-poor plasma” as used herein may refer to plasma from which most (e.g. at least 95%) or all of the platelets, and optionally most (e.g. at least 95%) or all of the cellular components, are removed. Platelet-poor plasma may obtained from a sample of whole blood, wherein the cellular components of the blood sample are separated from the liquid component (plasma) by an appropriate technique, typically by centrifugation (centrifugation step 1; e.g. 15 min at 1500 g at room temperature) and wherein subsequently the residual cellular components and/or platelets in the plasma are almost completely removed from the plasma by an appropriate technique, typically by centrifugation (centrifugation step 2; e.g. 15 min at 1500 g at room temperature). For example, platelet-poor plasma may contain at most 1.0×10⁴ platelets/pl. The term “platelet-free plasma” as used herein may refer to plasma from which all (i.e. at least 99.9%) of the platelets, and optionally all (i.e. at least 99.9%) of the cellular components, are removed.

In particular embodiments, the plasma sample comprises clotting factors and fibrinogen/fibrin.

In particular embodiments, the plasma sample is not subjected to an additional step of removing one or more of platelets, platelet fragments and residual blood cells (e.g. additional centrifugation step) from the plasma sample prior to step (c) (i.e. the step of determining the coagulation ability of said plasma sample). Indeed, the inventors have found that by using the method as disclosed herein (i.e. including the step of recovering of the plasma sample from the activated charcoal), no additional step of removing platelets or residual blood cells is required to allow accurate determination of the coagulation activity of the plasma.

In particular embodiments however, step (b) (i.e. the step of recovering the plasma sample from activated charcoal) does comprise passing said plasma sample through a filter. In particular embodiments, where step (b) comprises passing said plasma sample through a filter, the filter is preferably a filter with a pore size from 0.22 to 0.65 μm, such as a pore size of 0.45 μm. Preferably, in these embodiments the plasma sample is not subjected to yet an additional step (e.g. in addition to step (b)) of removing one or more of platelets, platelet fragments and residual blood cells (e.g. additional centrifugation step) from the plasma sample prior to step (c) (i.e. the step of determining the coagulation ability of said plasma sample).

In particular embodiments, the volume of the plasma sample which is contacted with the activated charcoal may be from 100 μl to 2000 μl, from 250 μl to 1500 μl, from 250 μl to 1000 μl, or from 500 μl to 1000 μl. Preferably, the volume of the plasma sample which is contacted with the activated charcoal is from 500 μl to 1000 μl.

Given that the methods of the invention are of particular interest in determining the health status of a subject, it is relevant that the plasma is a sample of the subject under consideration is only from said subject. While it can be of interest to mix the sample with a reference sample during the detection steps, this implies that the properties of the reference sample are known and the mixing step is relevant for the detection method.

The methods of the invention are of particular interest in diagnosing a haemostasis disorder in patients which are being treated to reduce the risk of stroke related to atrial fibrillation by blood thinners, more particularly patients that are being treated with direct anticoagulants. Accordingly, in particular embodiments, the subject or patient of which the plasma sample is obtained is selected from a patient group which was undergoing a treatment with a direct anticoagulant, preferably a DOAC, more preferably a DOAC selected from the list consisting of dabigatran etexilate, rivaroxaban, apixaban and edoxaban, prior to the in vitro diagnosis.

In particular embodiments, the method is envisaged for the in vitro diagnosis of a haemostasis disorder in a plasma sample of a subject or patient for which it is not known, more particularly at the time that coagulation ability needs to be determined, whether or not the patient has been treated with coagulation inhibitors, such as when the medical history of the subject or patient is unknown and/or cannot be established and/or cannot be ascertained. In particular embodiments, the method is envisaged for the in vitro diagnosis of a haemostasis disorder in a plasma sample of a subject or patient who is in trauma and/or unconscious.

In particular embodiments, the subject is a patient who has been treated with one or more direct anticoagulants, preferably a direct anticoagulant selected from the list consisting of betrixaban, argatroban, dabigatran etexilate, rivaroxaban, apixaban and edoxaban.

In particular embodiments, the subject is a patient who has been treated with one or more direct oral anticoagulant (DOAC), preferably a DOAC selected from the list consisting of dabigatran etexilate, rivaroxaban, apixaban and edoxaban.

It is the first time that it is demonstrated that the contacting of a plasma sample of a subject with activated charcoal results in a plasma sample no longer containing DOACs, to an extent which is sufficiently reliable to ensure detection of a blood haemostasis disorder in said subject. More particularly the methods of the present invention lead to a better efficiency of detecting anticoagulants and the ability to avoid false positives due to the presence of DOACs in a sample. The methods thus allow determining the coagulation ability of a plasma sample irrespective of the presence of DOACs in the sample.

The term “activated charcoal”, “activated carbon”, “active charcoal” or “active carbon” as used herein refers to microporous carbon. Microporous carbon may be obtained by processing carbon to increase the surface area thereof. Carbon with an increased surface area may be achieved by any method known in the art. A non-limiting example is the introduction of small, low-volume pores by chemically or physically (e.g. carbonization or oxidation) activating carbon. For example, 1 gram of activated carbon may have a surface are of at least 3000 m².

In particular embodiments, the activated charcoal is a powder. In more particular embodiments, the activated charcoal is a powder consisting of activated charcoal particles with an average size from 0.5 to 5 μm, from 1 to 4 μm, or from 2.5 to 3.5 μm. For example, activated charcoal particles with an average size of 3 μm.

The term “average size” as used herein refers to the average diameter if the activated charcoal particles are spherical and to the average volume-based particle size if the activated charcoal particles are non-spherical. The volume-based particle size equals the diameter of the sphere that has the same volume as a given particle. The volume-based particle size may be determined by any means known in the art to determine volume-based particle size of non-spherical particles, for example, using the formula: D=2*(3V/4π)^{1/3}; wherein D is the diameter of the representative sphere and V is the volume of the particle. In particular embodiments, the minimum diameter of the activated charcoal is at least 0.5 μm, at least 0.6 μm, at least 0.7 μm, at least 0.8 μm, at least 0.9 μm, at least 1 μm, at least 1.5 μm, at least 2 μm, or at least 2.5 μm.

In particular embodiments, the maximum diameter of the activated charcoal is at most 1000 μm, at most 500 μm, at most 250 μm, or at most 100 μm.

It will be understood that the absolute amount of activated charcoal to be used will depend on the size of the plasma sample. The average amount of activated charcoal will vary between 2 mg and 20 mg per milliliter of plasma. In particular embodiments, the plasma sample may be contacted (or incubated) with at least 2 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, or at least 20 mg of activated charcoal per milliliter of plasma. Preferably, the plasma sample may be contacted (or incubated) with at least 3 mg of activated charcoal per milliliter of plasma. More preferably, the plasma sample may be contacted with at least 5 mg of activated charcoal per milliliter of plasma.

In particular embodiments, the plasma sample may be contacted (or incubated) with from 2 to 20 mg of activated charcoal per milliliter of plasma, from 2 to 15 mg of activated charcoal per milliliter of plasma, from 5 to 15 mg of activated charcoal per milliliter of plasma, from 5 to 12 mg of activated charcoal per milliliter of plasma, from 8 to 12 mg of activated charcoal per milliliter of plasma, or from 9 to 11 mg of activated charcoal per milliliter of plasma. Preferably, the plasma sample is contacted (or incubated) with from 5 to 15 mg of activated charcoal per milliliter of plasma, such as 5 mg/ml, 6 mg/ml, 7 mg/ml, 8 mg/ml, 9 mg/ml, 10 mg/ml, 11 mg/ml; 12 mg/ml, 13 mg/ml, 14 mg/ml or 15 mg/ml. More preferably, the plasma sample is contacted (or incubated) with 10 mg of activated charcoal per milliliter of plasma.

In particular embodiments, the contacting step (or incubation step) may be performed during a period of at least 2 minutes, at least 3 minutes, at least 4 minutes, at least 5 minutes, at least 6 minutes, at least 7 minutes, at least 8 minutes, at least 9 minutes, or at least 10 minutes, preferably at least 2 minutes, more preferably at least 5 minutes.

The skilled person will understand that as the method as disclosed herein may be used in urgent situations, such as life threatening bleeding in unconscious or trauma patients, the steps of the method as disclosed herein are preferably as short as possible, while still providing a reliable result. Accordingly, it is envisaged that in particular embodiments, the plasma is contacted with the charcoal for 3 minutes prior to centrifugation.

The temperature at which the method is performed is not critical but is preferably around room temperature. Accordingly, in particular embodiments, the contacting step (or incubation step) may be performed at room temperature (i.e. ambient temperature).

The method is typically performed in a laboratory environment with sterilized material. Suitable tools for handling plasma samples are known in the art. In particular embodiments, the contacting step (or incubation step) may be performed in a container. Non-limiting examples of containers are Eppendorf tubes, multiwall plates, vials, spin filters and (centrifuge) tubes.

After having contacted the plasma sample with the charcoal, the charcoal is preferably removed from all or part of the sample so as to prevent interference of the charcoal in the further testing of the plasma sample. Accordingly, in particular embodiments, at least part, preferably all, of the plasma is recovered from the sample, i.e. removed from physical contact with the charcoal. In particular embodiments, the recovering of the plasma from the activated charcoal may comprise passing said plasma sample through a filter.

The term “filter” as used herein refers has its ordinary meaning in that it refers to a porous substance, device or membrane through which liquid is passed to remove suspended impurities or solid particles and/or to recover solids.

In the context of the invention, in particular embodiments, the filter is a membrane filter, such as a microporous plastic film.

The term “pore size” refers to the mean size of the pores on a membrane surface or filter. The pore size also relates to the filter's ability to filter out particles of a certain size. For example, a membrane filter with a pore size of 0.50 μm will filter out particles with a diameter of 0.50 μm or more from a filtration stream. Pore size may be determined by any methods known by the skilled person to determine pore size such as visual examination using scanning electron microscopy, porosimetry and/or particle challenge. Pores may be cylindrical or sponge pores.

In particular embodiments of the present invention, the recovering of the plasma from the activated charcoal may comprise passing said plasma sample through a filter with a pore size from 0.10 to 0.75 μm, from 0.20 to 0.70 μm, from 0.22 to 0.70 μm, from 0.22 to 0.65 μm, most preferably from 0.40 to 0.65 μm, from 0.50 to 0.65 μm, or from 0.60 to 0.65 μm. In particular embodiments, the filter has a pore size of 0.45 μm. Preferably, the recovering of the plasma from the activated charcoal may comprise passing said plasma sample through a filter with a pore size from 0.22 to 0.65 μm. For example, the recovering of the plasma from the activated charcoal may comprise passing said plasma sample through a filter with a pore size of 0.65 μm. In particular embodiments, the passing of the plasma sample through the filter may be achieved by any methods known by the skilled person. For example, the passing of the plasma sample through the filter may be achieved by gravity, vacuum, or pressure. Preferably, the passing of the plasma sample through the filter is achieved by centrifugation. Accordingly, in particular embodiments, the recovering of the plasma from the activated charcoal may comprise a centrifugation step and passing said plasma sample through a filter, preferably a filter with a pore size from 0.22 to 0.65 μm.

In particular embodiments, a centrifugation step is used to move plasma through a filter. In these embodiments, the duration of the centrifugation step may be from 2 to 10 minutes, from 2 to 7 minutes, or from 2 to 5 minutes. Preferably, the centrifugation step of from 2 to 5 minutes. Furthermore, the centrifugal force may be from 100 to 500 g, from 100 to 400 g, from 100 to 300 g, or from 100 to 200 g. Preferably, the centrifugation step is performed with a centrifugation force of 100 g.

In alternative embodiments, the recovering of the plasma from the activated charcoal may comprise a centrifugation step without the use of a filter. The centrifugation of the mixture of activated charcoal and plasma may separate the mixture into a plasma phase (i.e. upper phase) and an activated charcoal phase (i.e. lower phase and/or pellet). The plasma phase may then be physically removed from the centrifugation vial (e.g. by a pipette or an automatic syringe) to another container, to perform the coagulation testing.

The duration and centrifugal force of the centrifugation step for separating a sample into a plasma phase and a charcoal phase are known to the skilled person.

While it is envisaged that the methods of the invention remove the need for a separate removal of the platelets or other cells or fragments from the plasma sample, in particular embodiments, the methods can comprise an additional centrifugation step to remove substantially all platelets, platelet fragments, and/or blood cells from the plasma sample. If the centrifugation step is intended to remove substantially all platelets, platelet fragments, and/or blood cells from the plasma sample, the duration of the centrifugation step may be from 5 to 30 minutes, from 10 to 20 minutes, from 15 to 20 minutes. Additionally, the centrifugal force may be from 1000 g to 3000 g, from 1200 g to 1800 g, or from 1500 g to 1800 g.

According to the methods of the invention, the plasma obtained in step (b) does not comprise one or more direct anticoagulants. Preferably, and in particular in those embodiments where an appropriate filter and/or an additional centrifugation step is used, the plasma obtained after step (b) also does not comprise one or more of platelets, platelet fragments, residual blood cells. The skilled person will understand that the pore size of the filter will determine if platelet fragments will still be present in the plasma obtained in step (b). Where the method comprises passing said plasma sample through a filter with a pore size from 0.22 to 0.65 μm, the sample obtained will be substantially free of platelets (which typically have an average size from 0.5 to 2.5 μm), platelet fragments and residual blood cells (which typically have an average size from 6 to 14 μm), and further centrifugation is not required.

In line with the above, in particular embodiments, the method as disclosed herein does not comprise prior to the step of determining the coagulation ability of said plasma sample an additional step (e.g. in addition to step (b)) of removing (substantially all or all) of one or more of platelets, platelet fragments and residual blood cells from the plasma sample. More particularly, the methods do not comprise this additional centrifugation step if the step of recovering the plasma sample from activated charcoal comprises passing said plasma sample through a filter, preferably a filter with a pore size from 0.22 to 0.65 μm. For example, standard methods for obtaining platelet-free plasma starting from a whole blood sample typically comprise two centrifugation steps of at least 15 minutes (e.g. at 1500 g). In view hereof, the method as disclosed herein provides a faster approach to obtain platelet-free plasma, and allows removing all platelets and/or residual blood cells from the plasma sample instead of removing most of the platelets and/or residual blood cells in standard methods.

In particular embodiments, where the step of recovering the plasma sample from activated charcoal does not comprise passing said plasma sample through a filter with a pore size sufficiently small to remove platelets, platelet fragments and residual blood cells from the sample, the method as disclosed herein may comprise prior to step (c) (i.e. the step of determining the coagulation ability of said plasma sample) an additional step of removing one or more of platelets, platelet fragments and residual blood cells from the plasma, such as by centrifugation as described herein.

Given that the methods of the invention are intended to remove direct anticoagulants from the plasma, the methods do not require the use of universal or specific anticoagulation reversal agents to neutralize the anticoagulation agents present in the plasma. Accordingly, in particular embodiments, the method as disclosed herein does not comprise contacting the plasma sample with one or more universal or specific anticoagulant reversal agents either in step (c) or in the preparation of the sample for carrying out the coagulation assay.

The methods of the present invention allow for determining coagulation ability of a plasma sample without the potential interference of direct anticoagulants present in the sample. This is achieved by removing any direct anticoagulants that would be present by activated charcoal. The methods of the present invention allow for removing at least 80%, at least 90%, at least 95%, preferably at least 99% of the total amount of direct anticoagulants present in the sample; or removing substantially all direct anticoagulants present in the sample. The methods of the present invention allow for removing direct anticoagulants from the sample to a level of direct anticoagulants which is not capable of interfering with the in vitro diagnosis of a haemostasis disorder in the sample.

In particular embodiments, the methods as described herein allow for removing at least 100 ng of one or more direct anticoagulants per milliliter of plasma, at least 250 ng of one or more direct anticoagulants per milliliter of plasma, at least 500 ng of one or more direct anticoagulants per milliliter of plasma, at least 750 ng of one or more direct anticoagulants per milliliter of plasma, at least 1000 ng of one or more direct anticoagulants per milliliter of plasma, at least 1250 ng of one or more direct anticoagulants per milliliter of plasma, at least 1500 ng of one or more direct anticoagulants per milliliter of plasma, at least 2000 ng of one or more direct anticoagulants per milliliter of plasma, or at least 3000 ng of one or more direct anticoagulants per milliliter of plasma. Preferably, the methods as described herein allow for removing at least 1000 ng of one or more direct anticoagulants per milliliter of plasma.

In particular embodiments, the methods comprise, after having removed any potential direct anticoagulants from the plasma, the step of determining the coagulation ability of the plasma.

The term “coagulation ability” as used herein refers to the ability of the plasma to coagulate; and/or to the functionality and/or activity of one or more coagulation factors in the intrinsic and/or extrinsic coagulation pathways; optionally in the presence of one or more activators of the coagulation cascade. The step of determining the coagulation ability of a plasma sample may be carried out by any method known by the person skilled in the art to determine the coagulation ability. Non-limiting examples are coagulation assays, such as clot detection (e.g. by mechanical, photo-optical or viscoelastographic techniques), activated coagulation time, thrombin generation test, prothrombin time (PT), activated partial thromboplastin time (aPTT), lupus anticoagulant test, fibrinogen assays (both Clauss and PT derived-fibrinogen methods), thrombin (clot) time (TCT), specific factor activity assays (e.g. clotting assays or chromogenic assays for FVIII, FIX, X, XI, XII, XIII; VII, V, II or X), Proteins Induced by Vitamin K Antagonism or Absence (PIVKA) test or thrombotest, activated protein C resistance (APCR) assay, Protein C activity assay, Protein S activity assay, antithrombin activity assay and thrombin generation assay and dilute Russell Viper Venom Test/Time (dRVVT).

In particular embodiments, the step of determining of the coagulation ability of said plasma sample comprises one or more optic, immunologic, chromogenic and/or fluorogenic coagulation assays.

In particular embodiments, the step of determining the coagulation ability of the plasma sample comprises determining the ability of the plasma sample to form a clot; optionally in the presence of one or more activators of the coagulation cascade. The clot formation may be measured optically or mechanically. The inability of said plasma sample to clot is indicative of the presence, progression, or severity of a haemostasis disorder in said subject, and optionally of the nature of the haemostasis disorder.

In particular embodiments, the step of determining the coagulation ability of the plasma sample comprises determining the ability of the plasma sample to normalize the prolonged clotting time of specific factor-deficient plasma. The inability of said plasma sample of a subject to normalize the prolonged clotting time of specific factor-deficient plasma is indicative of the presence, progression, or severity of a haemostasis disorder in said subject, and optionally of the nature of the haemostasis disorder.

In particular embodiments, the step of determining the coagulation ability of the plasma sample comprises assessing the ability of a specific coagulation factor to cleave a fluorogenic/chromogenic-linked substrate. The inability of said plasma sample to cleave a fluorogenic/chromogenic-linked substrate is indicative of the presence, progression, or severity of a haemostasis disorder in said subject, and optionally of the nature of the haemostasis disorder.

In particular embodiments, the step of determining the coagulation ability of said plasma sample obtained under step (b) may be performed by contacting the plasma sample with a coagulation activator.

In particular embodiments, the coagulation activator is selected from the group consisting of human calcium thrombin, rabbit or recombinant human tissue factor, synthetic phospholipids, Russel's viper venom, ecarin, textarin or silica, colloidal silica activator, thrombomodulin, activated protein C, lyophilized bovine thrombin and chromogenic substrate of thrombin CBS 61.50, factor V activator from snake venom and factor Va-dependent prothrombin activator isolated from snake venom.

In particular embodiments, the step of determining the coagulation ability of said plasma sample obtained under step (b) further comprises contacting said plasma sample with an immune depleted serum or plasma prior to step (c). In particular embodiments, said immune depleted serum or plasma is selected from the group consisting of Factor VIII or IX or X or XI or XII or XIII or VII or V or II deficient serum or plasma.

In further particular embodiments, the step of determining the coagulation ability of said plasma sample comprises one or more of determining prothrombin time, activated partial thromboplastin time, thrombin time or fibrinogen, activated protein C resistance assessment, performing a thrombin generation assay, lupus anticoagulant testing, or protein C, S and antithrombin measurements.

For instance, Lupus anticoagulants (LA) are classified as antiphospholipid antibodies (APA), although they are in fact directed against phospholipid-binding proteins, in particular, β2 glycoprotein I and prothrombin. The presence of persistent LA has a greater association with thrombosis, pregnancy morbidity and recurrence than the criteria antibodies detected in solid phase assays (aCL & aβ2GPI). LA are a heterogeneous group of autoantibodies that can be detected by inference based on their behaviour in phospholipid-dependent coagulation assays. However, this requires that other possible causes of elevated clotting times have been excluded. In particular embodiments, the step of determining the coagulation ability of said plasma sample is performed by a coagulation test chosen from the list comprising prothrombin time (PT), activated thromboplastin time (aPTT), lupus anticoagulant test, fibrinogen assays (both Clauss and PT derived-fibrinogen methods), thrombin time, coagulation factor activity assays (FVIII, FIX, X, XI, XII, XIII, VII, V, II, X), activated protein C resistance (APCR) assay, Protein C activity assay, Protein S activity assay, antithrombin activity assay and thrombin generation test. Indeed, in particular embodiments, the step of determining the coagulation ability of the plasma sample comprises determining whether the sample is capable of correcting immune-depleted plasma, by contacting said sample with one or more types of immune depleted plasma selected from Factor VIII or IX or X or XI or XII or XIII or VII or V or II-depleted plasma.

In particular embodiments, the step of determining the coagulation ability of said plasma sample is determined using a blood clotting-based method for determining Fibrinogen deficiency, Prothrombin deficiency, Factor V deficiency, Factor V Leiden, Protein C deficiency, protein S deficiency, antiplasmin deficiency, antithrombin deficiency, plasminogen deficiency, Elevated D-Dimer, antiphospholipid syndrome, heparin induced thrombocytopenia, Combined Factor V and VIII deficiency, Factor VII deficiency, Factor VIII deficiency (Haemophilia A), Factor IX deficiency (Haemophilia B), Factor X deficiency, Factor XI deficiency, Factor XIII deficiency, Glanzmann's thrombasthenia, Bernard Soulier Syndrome, Wiskott-Aldrich Syndrome or Leukocyte Adhesion deficiency, and said method further provides an indication of the nature of said haemostasis disorder.

In particular embodiments, the step of determining the coagulation ability of said plasma sample as described in the method as disclosed herein, is performed using one of the following test, or a combination thereof:
A quantitative determination of fibrinogen, preferably by adding or mixing an excess of lyophilized human calcium thrombin with the plasma obtained in step (b).
A thrombin time (TT) test, preferably by adding or mixing lyophilized human calcium thrombin with the plasma obtained in step (b).
A prothrombin time (PT) test, preferably by adding or mixing rabbit or recombinant human tissue factor, synthetic phospholipids and stabilizers with the plasma obtained in step (b).
A determination of the activated partial thromboplastin time (aPTT), preferably by adding or mixing synthetic phospholipid reagent containing a colloidal silica activator with the plasma obtained in step (b).
The detection of a lupus anticoagulant in plasma (using either the dilute Russell's viper venom time, the textarin, the ecarin or the aPTT methods), preferably by adding or mixing Russel's viper venom, ecarin, textarin or silica, phospholipids and calcium with the plasma obtained in step (b).
The determination of factor VIII activity in plasma, preferably by adding or mixing lyophilized citrated human plasma from which factor VIII has been removed by immune-adsorption with the plasma obtained in step (b).
The determination of factor IX activity in plasma, preferably by adding or mixing lyophilized citrated human plasma from which factor IX has been removed by immune-adsorption with the plasma obtained in step (b).
The determination of factor XI activity in plasma, preferably by adding or mixing lyophilized citrated human plasma from which factor XI has been removed by immune-adsorption with the plasma obtained in step (b).
The determination of factor XII activity in plasma, preferably by adding or mixing lyophilized citrated human plasma from which factor XII has been removed by immune-adsorption with the plasma obtained in step (b).
The determination of factor VII activity in plasma, preferably by adding or mixing lyophilized citrated human plasma from which factor VII has been removed by immune-adsorption with the plasma obtained in step (b).
The determination of factor V activity in plasma, preferably by adding or mixing lyophilized citrated human plasma has been artificially depleted for factor V with the plasma obtained in step (b).
The determination of factor II activity in plasma, preferably by adding or mixing lyophilized citrated human plasma has been artificially depleted for factor II with the plasma obtained in step (b).
The determination of the kaolin-activated partial thromboplastin time (aPTT), preferably by adding or mixing cephalin and factor XII activator, kaolin, with the plasma obtained in step (b).
The determination of resistance to activated protein C caused by the factor V Leiden mutation, preferably by adding or mixing factor V activator from snake venom and factor Va-dependent prothrombin activator isolated from snake venom with the plasma obtained in step (b).
The quantitative determination of the antithrombin activity level, preferably by adding or mixing lyophilized bovine thrombin and chromogenic substrate of thrombin CBS 61.50 with the plasma obtained in step (b).

In particular embodiments, the methods of the invention allow a reduction in the controls required for determining the coagulation ability of said plasma sample. Indeed, in practice, most particularly where this cannot be confirmed by the patient, additional tests are typically required to exclude influence of anti-coagulants on the results obtained. Accordingly, in particular embodiments, the methods of the invention comprise determining the coagulation ability of said plasma using only one or a limited number of assays.

In particular embodiments, the methods of the invention allow a representative measurement of the anticoagulant factors in the plasma of a patient. Indeed, removal of DOACs according to the invention allow for representative results using well-established assays.

For instance, in particular embodiments, the step of determining the coagulation ability of said plasma sample comprises the detection of a lupus anticoagulant in plasma. Generally, testing of lupus anticoagulant requires screening, confirmatory and mixing tests. Screening tests commonly employ dilute phospholipid to accentuate the in vitro anticoagulant effect of LA, which if present, will prolong the clotting time. However, screening tests can be prolonged for reasons other than LA, (i.e. factor deficiencies, anticoagulant therapy), so all elevated screening tests require follow-up analyses to help define the nature of any abnormality. The confirm test generally involves performing the screening test in an identical fashion except that the phospholipid concentration is markedly increased. In particular embodiments, the lupus anticoagulant testing is selected from dilute Russell's viper venom time (dRVVT), LA-responsive APTT or combinations thereof. In particular embodiments, the lupus anticoagulant testing comprises the use of a dilute APTT (dAPTT) in which employs a silica activator and a low concentration of phospholipid comprised of a composition of phospholipid types that is LA-responsive. Prior removal of DOAC according to the invention will allow representative detection of LA in the sample using these methods.

By reversing the effect of direct anticoagulants, the method as disclosed herein can be used in the diagnosis of haemostasis disorders in patients which have been treated with oral or parental direct anticoagulants. Hence, the method as disclosed herein allows to easily determine whether the observed lack of blood clotting can be attributed to a haemostasis disorder or the presence of direct anticoagulants in the plasma sample from said patient.

Coagulation tests or assays most affected by the presence of anticoagulants in the plasma sample will be the tests that involve the clotting factor to which the coagulation inhibitor is directed, leading to false-positive or false-negative results (Table 1).

TABLE 1
Interference of direct oral anticoagulants with various coagulation function assessments

| Test | Dabigatran | Rivaroxaban | Apixaban | Edoxaban | Betrixaban | Notes |
|---|---|---|---|---|---|---|
| PT/clotting | ↓ | ↓↓/↓↓↓ | ↓/↓↓ | ↓↓ | ↓↓ | All factors |
| factors | | | | | | affected |
| | | | | | | Most sensitive |
| | | | | | | to rivaroxaban |
| aPTT/clotting | ↓↓↓ | ↓↓ | ↓/↓↓ | ↓/↓↓ | ↓↓ | All factors |
| factors | | | | | | affected |
| | | | | | | Most sensitive |
| | | | | | | to dabigatran |
| Lupus | ↑/↑↑ | ↑/↑↑ | —/↑ | ↑/↑↑ | ND | False positive |
| anticoagulant | | | | | | due to high |
| | | | | | | screen/confirmation |
| | | | | | | assay ratios |
| APCR | ↑ | ↑ | ↑ | ↑ | ND | aPTT-based |
| | | | | | | assays are |
| | | | | | | mostly affected |
| | | | | | | No interference |
| | | | | | | of rivaroxaban |
| | | | | | | with Pefakit |
| | | | | | | APCR Factor V |
| | | | | | | Leiden ® |
| Protein C | —/↑ | —/↑ | —/↑ | — | ND | Chromogenic |
| activity | | | | | | assays: |
| | | | | | | unaffected |
| | | | | | | Antigen-based |
| | | | | | | assays: |
| | | | | | | unaffected |
| | | | | | | Clot-based |
| | | | | | | assays: affected |
| Protein S | —/↑ | —/↑ | —/↑ | ↑ | ND | Antigen-based |
| activity | | | | | | assay: |
| | | | | | | unaffected |
| | | | | | | Clot- |
| | | | | | | based: affected |
| Antithrombin | —/↑ | —/↑ | —/↑ | —/↑ | ND | Anti-thrombin- |
| activity | | | | | | based assays |
| | | | | | | are affected by |
| | | | | | | dabigatran |
| | | | | | | Anti-Factor Xa- |
| | | | | | | based assays |
| | | | | | | are affected by |
| | | | | | | the Factor Xa |
| | | | | | | inhibitors |

APCR, activated protein C resistance;
aPTT, activated partial thromboplastin time;
PT, prothrombin time;
ND, not done

The use of the method as disclosed herein, preferably wherein the step of recovering the plasma sample from the activated charcoal comprises passing said plasma sample through a filter with a pore size from 0.22 to 0.65 μm, allows an increase in the reliability of the diagnosis of a hemostasis disorder and more particularly allows the differentiation between a decreased coagulation ability (e.g. lack of blood clotting) due to a haemostasis disorder or due to the presence of (direct) coagulation inhibitors in the plasma sample from said patient. The use of the method as disclosed herein, preferably wherein the step of recovering the plasma sample from the activated charcoal comprises passing said plasma sample through a filter with a pore size from 0.22 to 0.65 μm, enables the differentiation of a decreased coagulation ability related to antithrombotic therapy from another aetiology.

The use of the method as disclosed herein, preferably wherein the step of recovering the plasma sample from the activated charcoal comprises passing said plasma sample through a filter with a pore size from 0.22 to 0.65 μm, is of particular interest for the analysis of a sample of a patient for which it is not known whether or not the patient has been treated with (direct) coagulation inhibitors, such as in situations where the patient is not able to provide said information. Indeed, as the invention ensures that the prior treatment of the patient with (direct) coagulation inhibitors does not affect the diagnosis, this avoids the risk of an incorrect diagnosis when the prior treatment of the patient is unknown. Accordingly, in particular embodiments of the method as provided herein, the medical history of the patient is unknown. Additionally, the present invention is of interest where the patient has been treated with (direct) coagulation inhibitors.

A further aspect relates to a diagnostic kit, such as for the in vitro diagnosis of a haemostasis disorder and/or for preparing a plasma sample for the in vitro diagnosis of a haemostasis disorder comprising
activated charcoal;
a vial comprising a filter, preferably a filter with a pore size from 0.22 to 0.65 μm;
   and optionally one or more compounds required for the in vitro diagnosis of a haemostasis disorder. In particular embodiments, the diagnostic kit may comprise from 2 mg to 20 mg, from 2 mg to 10 mg, from 2.5 mg to 7.5 mg, or from 2.5 mg to 5 mg of activated charcoal per vial. Preferably, the diagnostic kit, more particularly the vial provided therein, comprises from 2.5 mg to 10 mg of activated charcoal per vial. In particular embodiments, the vial contains 4 to 8 mg of activated charcoal, such as 5 to 7 mg of activated charcoal.

In particular embodiments, the vial may have a volume from 100 μl to 10000 μl, from 100 μl to 5000 μl, from 250 μl to 2500 μl, from 250 μl to 2000 μl, from 250 μl to 1500 μl, or from 500 μl to 1000 μl. Preferably, the vial has a volume from 100 μl to 1000 μl, such as but not limited to 500 μl to 1000 μl. Indeed, the invention particularly envisages the use of the method in the analysis of patient samples, which typically involve the collection of a limited amount of blood, such as in vials of between 100 μl to 10000 μl, such as vials of 500 μl.

In particular embodiments, the filter may have a pore size from 0.10 to 0.75 μm, from 0.20 to 0.70 μm, from 0.22 to 0.70 μm, from 0.22 to 0.65 μm, from 0.40 to 0.65 μm, from 0.50 to 0.65 μm, or from 0.60 to 0.65 μm. Preferably, the filter has a pore size from 0.22 to 0.65 μm, such as a pore size of 0.45 μm

In particular embodiments, the filter is positioned within a filter device which is suitable for placement in a vial, whereby upon centrifugation of the vial, the fluid placed within the filter device passes through the filter into the vial. In particular embodiments, the filter device is suitable for placement in an Eppendorf tube of 250 μl-2000 μl.

In particular embodiments, the filter device further comprises charcoal. In further embodiments, the filter device comprises 5 to 7 mg of charcoal.

In particular embodiments, the vial may be a vacutainer or an Eppendorf tube.

In particular embodiments, the one or more compounds required for the in vitro diagnosis of a haemostasis disorder are one or more activators of coagulation cascade and/or immune deficient plasma.

The diagnostic kit may further comprise ready-to use substrate solutions, wash solutions, dilution buffers and additional compounds (e.g. phospholipids, snake venoms, calcium, calcium chloride, tissue factor, silica, celite, kaolin, ellagic acid, coagulation factors from human or animal origin). The diagnostic kit may also comprise positive and/or negative control samples. For example, solubilised coagulation inhibitors, preferably thrombin and factor Xa inhibitors, more preferably dabigatran etexilate, rivaroxaban, apixaban or edoxaban.

The one or more compounds for the in vitro diagnosis of a haemostasis disorder are compounds which allow the detection of a haemostasis disorder. In particular embodiments, the one or more compounds for the in vitro diagnosis of a haemostasis disorder include a coagulation activating agent. In particular embodiments, the diagnostic kit as disclosed herewith comprises compounds necessary for performing any of the following tests or combinations thereof:
A quantitative determination of fibrinogen, preferably by adding or mixing an excess of lyophilized human calcium thrombin to the plasma obtained in step (b).
A thrombin time (TT) test, preferably by adding or mixing lyophilized human calcium thrombin with the plasma obtained in step (b).
A prothrombin time (PT) test, preferably by adding or mixing rabbit or recombinant human tissue factor, synthetic phospholipids and stabilizers with the plasma obtained in step (b).
A determination of the activated partial thromboplastin time (APTT), preferably by adding or mixing synthetic phospholipid reagent containing a colloidal silica activator with the plasma obtained in step (b).
The detection of a lupus anticoagulant in plasma (using either the dilute Russell's viper venom time, the textarin, the ecarin or the aPTT methods), preferably by adding or mixing Russel's viper venom, ecarin, textarin or silica, phospholipids and calcium with the plasma obtained in step (b).
The determination of factor VIII activity in plasma, preferably by adding or mixing lyophilized citrated human plasma from which factor VIII has been removed by immune-adsorption with the plasma obtained in step (b).
The determination of factor IX activity in plasma, preferably by adding or mixing lyophilized citrated human plasma from which factor IX has been removed by immune-adsorption with the plasma obtained in step (b).
The determination of factor XI activity in plasma, preferably by adding or mixing lyophilized citrated human plasma from which factor XI has been removed by immune-adsorption with the plasma obtained in step (b).
The determination of factor XII activity in plasma, preferably by adding or mixing lyophilized citrated human plasma from which factor XII has been removed by immune-adsorption with the plasma obtained in step (b).
The determination of factor VII activity in plasma, preferably by adding or mixing lyophilized citrated human plasma from which factor VII has been removed by immune-adsorption with the plasma obtained in step (b).
The determination of factor V activity in plasma, preferably by adding or mixing lyophilized citrated human plasma has been artificially depleted for factor V with the plasma obtained in step (b).
The determination of factor II activity in plasma, preferably by adding or mixing lyophilized citrated human plasma has been artificially depleted for factor II with the plasma obtained in step (b).
The determination of the kaolin-activated partial thromboplastin time (APTT), preferably by adding or mixing cephalin and factor XII activator, kaolin, with the plasma obtained in step (b).
The determination of resistance to activated protein C caused by the factor V Leiden mutation, preferably by adding or mixing factor V activator from snake venom and factor Va-dependent prothrombin activator isolated from snake venom with the plasma obtained in step (b).
The quantitative determination of the antithrombin activity level, preferably by adding or mixing lyophilized bovine thrombin and chromogenic substrate of thrombin CBS 61.50 with the plasma obtained in step (b).

In particular embodiments, the diagnostic kit may comprise carriers which allow visualization and/or a qualitative read-out of the coagulation ability of the plasma sample of the patient by, for example, spectrophotometry or mechanical clotting detection.

The term ‘carrier’ as used herein refers to small containers in which the clotting reaction is performed. Typically, the minimum volume that the container can hold is larger than the minimum total volume required for the clotting reaction to occur. Optionally, these carriers may also allow for cascade testing. Non-limiting examples of carriers are translucent microtiter plates, translucent stripwells or translucent tubes.

Preferably, the instructions included in the diagnostic kit are unambiguous, concise and comprehensible to those skilled in the art. The instructions typically provide information on kit contents, how to obtain the plasma sample, methodology, experimental read-outs and interpretation thereof and cautions and warnings.

The present invention is further illustrated in the following non-limiting examples.

### EXAMPLES

### Example 1: Influence of DOACs Present in a Serum Sample on aPTT and PT Assays for In Vitro Diagnosis of a Haemostasis Disorder

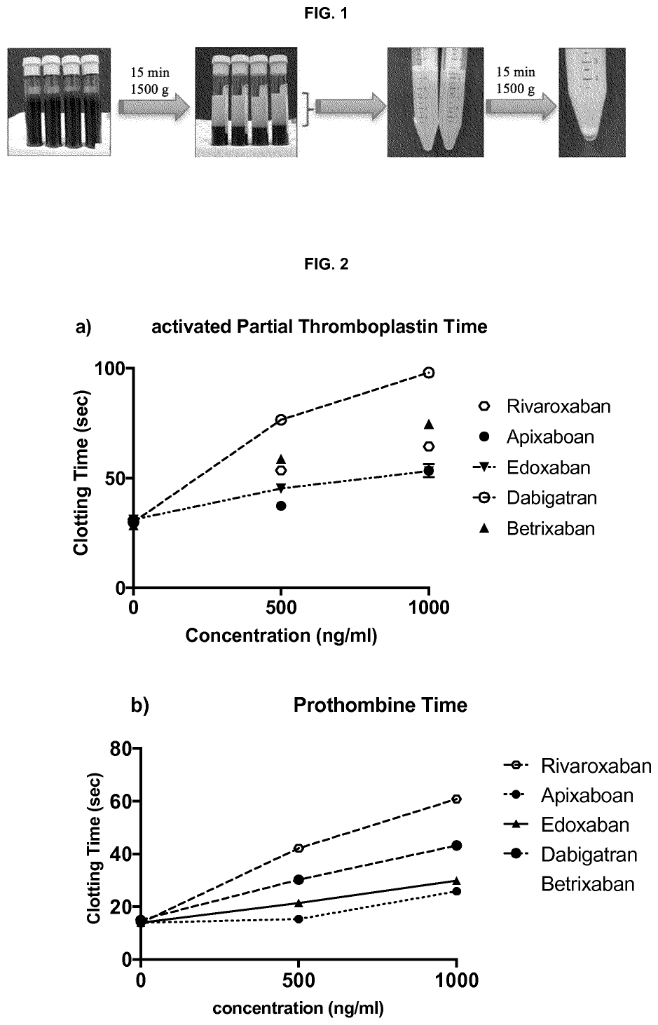

Standard tests for in vitro diagnosis of a haemostasis disorder, for example in hospital laboratories, are generally performed on plasma samples obtain from a patient. For the preparation of such plasma samples, blood components of a blood sample from a patient are typically separated by two centrifugation steps, both at 2500 g for 15 min (FIG. 1). The first centrifugation step will separate the blood in solid substances (e.g. red and white blood cells) (i.e. the lower phase) and blood plasma (i.e. upper phase). Subsequently, the blood plasma is collected and submitted to a second centrifugation step, which pelletizes the residual blood cells and/or platelets. The upper phase obtained by the second centrifugation step (i.e. platelet-poor plasma) can be used for haemostatic tests. However, the platelet-poor plasma obtained by standard methods will comprise direct anticoagulants (e.g. DOACs), if the patient is treated therewith, which may interfere with several clotting times tests (Table 1).

DOACs, such as Rivaroxaban, Apixaboan, Edoxaban, Dabigatran and Betrixaban, prolonged the activated Partial Thromboplastin time (aPTT) (FIG. 2a) and prothrombin time (PT) (FIG. 2b) of platelet-poor plasma (i.e. plasma obtained after the second centrifugation step). Furthermore, the prolongation of the clotting time is proportional to the concentration of DOACs.

### Example 2: The Method as Disclosed Herein Removes the Influence of DOACs on aPTT and PT Assays for the In Vitro Diagnosis of a Haemostasis Disorder

A blood sample obtained from a subject was centrifuged at 2500 g during 15 min to separate the blood into solid substances (e.g. red and white blood cells) (i.e. lower phase) and blood plasma (i.e. upper phase).

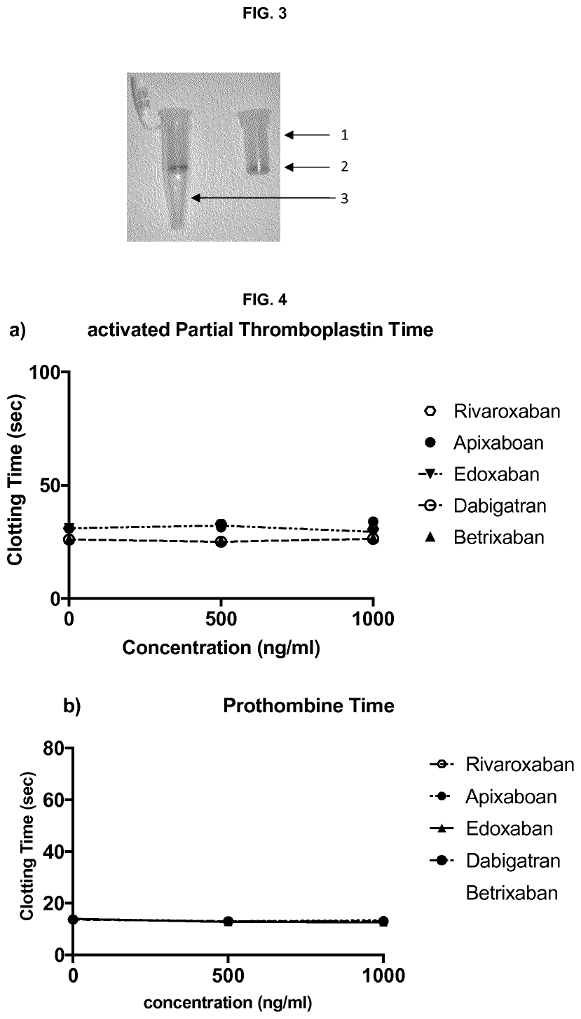

The plasma obtained from the first (and only) centrifugation step was incubated with activated charcoal (10 mg/ml of plasma) for 5 minutes and the plasma was subsequently recovered from the activated charcoal by passing the plasma through a filter with 0.65 μm pores (FIG. 3). Passing the plasma through the filter was achieved by a short centrifugation. The filter with 0.65 μm pores allowed to efficiently remove the activated charcoal and residual blood cells and/or platelets, which have a larger size than 0.65 μm, from the plasma with a minimal interference with blood coagulation tests.

Accordingly, it appears that recovering the plasma from the activated charcoal by passing the plasma through a filter replaces the second centrifugation step to obtain platelet-poor plasma as disclosed in Example 1.

The filtered plasma was used in aPTT and PT assays. The results hereof show that the effect of DOACs on the aPTT and PT assays was completely removed, even at high concentrations of DOACs, such as 1000 ng/ml (FIGS. 4a and 4b), by the method as disclosed herein. In view hereof, the method as disclosed herein allows exploring the coagulation cascade in patients treated with DOACs and provides a reliable assessment of the coagulation ability of a plasma sample.

### Example 3: Effect of the Concentration of Activated Charcoal on the Elimination of the Influence of DOACs on aPTT and PT Assays

Plasma was obtained by centrifuging once a blood sample obtained from a subject as described in Example 2. The plasma sample was incubated with different concentrations of activated charcoal (i.e. 5 mg/ml, 10 mg/ml or 15 mg/ml) for 5 minutes and the plasma was subsequently recovered from the activated charcoal by passing the plasma through a filter with 0.65 μm pores and activated charcoal by a short centrifugation.

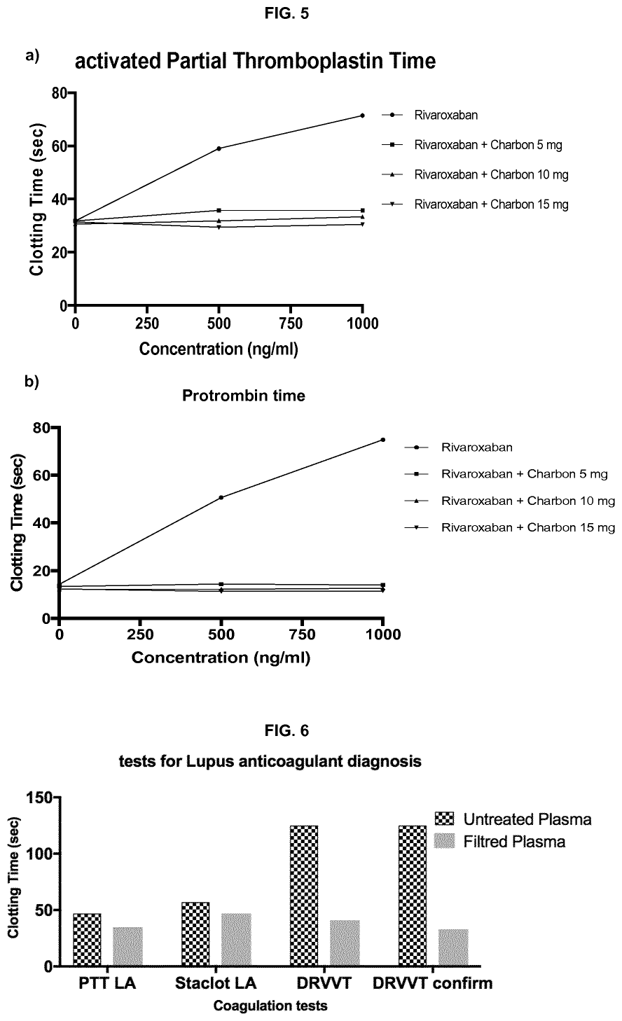

The filtered plasma sample was used in aPTT and PT assays. The results hereof show that the effect of Rivaroxaban on the aPTT and PT assays was completely removed, even at high concentrations of Rivaroxaban, such as 1000 ng/ml (FIGS. 5a and 5b). Furthermore, 5 mg of activated charcoal/ml was already sufficient to obtain this effect.

### Example 4: The Method as Disclosed Herein Removes the Influence of DOACs on Coagulation Tests in a Clinical Setting

The efficacy of the method as disclosed herein was assessed in a clinical situation. FIG. 6 shows the results for a plasma sample obtained from a patient treated with rivaroxaban (plasma sample comprised 339 ng rivaroxaban per ml of plasma) who was suspected to suffer from lupus anticoagulant (LA), which is a pro-thrombotic disease. For LA diagnosis, several in vitro diagnosis assays were performed on the patient's plasma, including Partial Thromboplastin Time-Lupus anticoagulant Screen (PTT LA), PTT LA confirm (Staclot LA), Dilute Russel Viper Venom Time screen (DRVVT) and DRVVT confirm.

FIG. 6 shows that the clotting times for untreated plasma (i.e. not treated by the method as disclosed herein; platelet-poor plasma as obtained in Example 1) were prolonged for all LA diagnostic assays, especially for DRVVT and DRVVT confirm. Accordingly, it could be concluded from these tests that the patient has a LA.

On the other hand, when plasma treated by the method as disclosed herein (i.e. obtained as in Example 2) was used in the LA diagnostic assays, the results clearly show that the clotting times are within normal ranges. Accordingly, it could be concluded from the tests using plasma treated by the method as disclosed herein that the patient does not have LA.

In view of the above, it appears that the presence of rivaroxaban in the patient's plasma prolongs the clotting time for all LA diagnostic assays and this could lead to a false diagnosis. Treatment of plasma by the method as disclosed herein replaces the second centrifugation step to obtain platelet-poor plasma as disclosed in Example 1, and is therefore a shorter method than the standard method. Furthermore, the method as disclosed herein allows removing the effect of the presence of DOACs on clotting times in LA diagnostic assays, such as PTT LA, Staclot LA, DRVVT and DRVVT confirm.

### Example 5: Influence of DOACs Present in a Plasma Sample on the In Vitro Determination of Lupus Anticoagulant

LA detection involves use of screening, mixing tests and confirmatory. Screening tests commonly employ low phospholipids content reagents to accentuate the in vitro anticoagulant effect of LA, which if present, will prolong the clotting time. Screening tests can be prolonged for reasons other than LA, (i.e. factor deficiencies, anticoagulant therapy), so all elevated screening tests receive follow-up analyses to help define the nature of any abnormality. The confirm test generally involves performing the screening test in an identical environment except that the phospholipid concentration is markedly increased. This has the effect of partially or completely overwhelming the LA and thus leads to a shorter clotting time than the screening test, thereby evidencing phospholipid dependence. Clotting times are converted to ratios to mitigate for issues of analytical variability. Correction of the screen ratio by the confirm ratio by ≥10% is considered consistent with the presence of a LA, providing that other causes of elevated clotting times are excluded.

Diagnostic specificity is improved by performing the screen and confirmatory tests on 1:1 mixtures of test and normal plasma to evidence inhibition and reduce interferences, although the inevitable dilution effect can compromise this aspect of analysis. Antibody heterogeneity and reagent variability necessitate use of at least two assays, of different analytical principle, to achieve acceptable detection rates. First-line assays are dilute Russell's viper venom time (dRVVT) in combination of a LA-sensitive APTT (PTT-LA), a pairing that will detect most clinically significant antibodies.

For the testing of LA in a number of samples in the laboratory, a combination of dRVVT and a LA-sensitive APTT was used, which employs a silica activator and a low concentration of phospholipid comprised of a composition of phospholipid types that is LA-sensitive. The confirm test involved addition of concentrated platelet-derived phospholipid. For the dRVVT analysis, diluted FX activator from the venom of Russell's viper (Daboia russellii), a low concentration of phospholipid comprised of a composition of phospholipid types that is LA-responsive and calcium ions was used. The confirm test involves an identical reagent except that the same phospholipid preparation is employed at a higher concentration. All elevated APTT & dRVVT screen results are reflexed to receive the confirm test, and the screen and confirm mixing tests, and are reported with interpretive comment. Patients with LA may be positive in one or both of the PTT-LA & dRVVT test medleys.

To show the interference of DOACs on LA testing, a normal pooled plasma (NPP) from healthy donors was spiked with either dabigatran, apixaban, rivaroxaban or edoxaban at final concentrations of 0-100-300-1000 ng/ml. Two conditions were then tested: i) the spiked NPP was tested directly for both dRVVT-screen/confirm and PTT-LA or ii) it was incubated in the device (which contains the active charcoal at the amount of 5 to 7 mg/filter) for 5 minutes and then filtrated by a centrifugation step set at 200 g during 2 minutes. Plasma collected in vial is then depleted of DOACs and can also be tested for DRVVT screen/confirm and PTT-LA assays without any influence from DOACs.

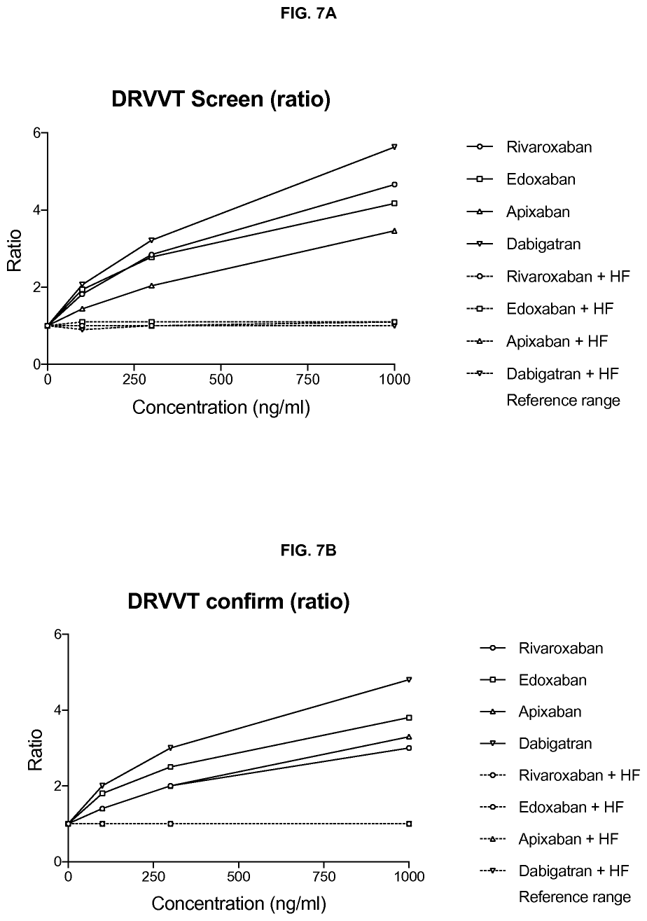

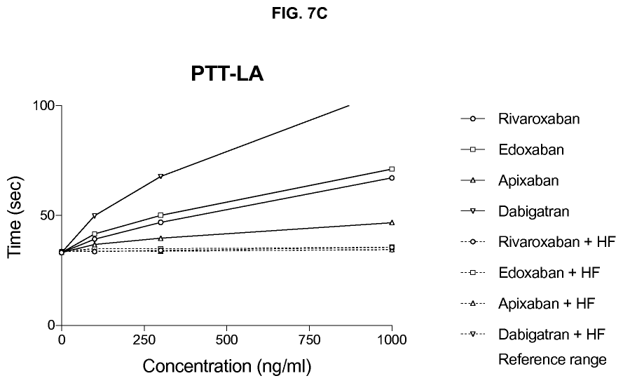

The results of the two conditions mentioned above are shown in FIG. 7. These results show that plasma samples containing DOACs showed prolonged clotting times that are out of reference range for all the investigated clotting tests, leading to false positive results. These samples return to the reference range when DOACs are removed according to the invention. These results demonstrate that false positives for LA diagnosis due to the presence of DOAC are avoided. Thus, first-line assays are impacted by the presence of DOACs, and the removal of DOACs allows the recovery of the baseline value (i.e. when the NPP is not spiked with any DOACs) as described in FIGS. 7A and 7B for dRVVT screen/confirm ratio and in FIG. 7C for LA sensitive APTT.

1. A method for in vitro diagnosis of a haemostasis disorder in a plasma sample obtained from a subject comprising the steps of
contacting a plasma sample obtained from said subject with activated charcoal;
recovering said plasma sample from said activated charcoal;
determining the coagulation ability of said plasma sample recovered from said activated charcoal; and
determining, based on said coagulation ability of said plasma, the presence, progression, or severity of a haemostasis disorder in said subject, and optionally the nature of the haemostasis disorder.

2. The method according to claim 1, wherein the step of recovering said plasma sample from said activated charcoal comprises passing said plasma sample through a filter with a pore size from 0.22 μm to 0.65 μm.

3. The method according to claim 1, wherein the step of recovering said plasma from said activated charcoal comprises a centrifugation step.

4. The method according to claim 1, wherein said activated charcoal has a concentration of at least 3 mg/ml.

5. The method according to claim 1, wherein said plasma sample is contacted with activated charcoal for at least 2 minutes.

6. The method according to claim 1, which does not comprise an additional step of removing platelets, platelet fragments or residual blood cells, or a combination thereof from said plasma sample prior to determining the coagulation ability of said plasma sample.

7. The method according to claim 1, wherein the step of determining the coagulation ability of said plasma sample recovered from said activated charcoal is performed by contacting said plasma sample with a coagulation activator.

8. The method according to claim 7, wherein the coagulation activator is selected from the group consisting of human calcium thrombin, rabbit or recombinant human tissue factor, synthetic phospholipids, Russel's viper venom, ecarin, textarin or silica, colloidal silica activator, thrombomodulin, activated protein C, lyophilized bovine thrombin and chromogenic substrate of thrombin CBS 61.50, factor V activator from snake venom and factor Va-dependent prothrombin activator isolated from snake venom.

9. The method according to claim 1, wherein the step of determining the coagulation ability of said plasma sample recovered from said activated charcoal further comprises contacting said plasma sample with an immune depleted serum or plasma prior to determining the coagulation ability of said plasma sample.

10. The method according to claim 9, wherein the immune depleted serum or plasma is selected from the group consisting of Factor VIII or IX or X or XI or XII or XIII or VII or V or II deficient serum or plasma.

11. The method according to claim 1, wherein the step of determining the coagulation ability of said plasma sample recovered from said activated charcoal is performed by a coagulation test comprising prothrombin time (PT), activated partial thromboplastin time (aPTT), lupus anticoagulant test, a fibrinogen assay, thrombin time, coagulation factor activity assays (FVIII, FIX, X, XI, XII, XIII; VII, V, II, X), activated protein C resistance (APCR) assay, Protein C activity assay, Protein S activity assay, antithrombin activity assay, or thrombin generation assay, or a combination thereof.

12. The method according to claim 1,
wherein the step of determining the coagulation ability of said plasma sample recovered from said activated charcoal comprises using a blood clotting-based method for determining Fibrinogen deficiency, Prothrombin deficiency, Factor V deficiency, Factor V Leiden, Protein C deficiency, protein S deficiency, antiplasmin deficiency, antithrombin deficiency, plasminogen deficiency, Elevated D-Dimer, antiphospholipid syndrome, heparin induced thrombocytopenia, Combined Factor V and VIII deficiency, Factor VII deficiency, Factor VIII deficiency (Haemophilia A), Factor IX deficiency (Haemophilia B), Factor X deficiency, Factor XI deficiency, Factor XIII deficiency, Glanzmann's thrombasthenia, Bernard Soulier Syndrome, Wiskott-Aldrich Syndrome, or Leukocyte Adhesion deficiency, and
wherein said method further provides an indication of the nature of said haemostasis disorder.

13. The method according to claim 1, wherein the step of determining the coagulation ability of said plasma sample recovered from said activated charcoal is performed by a lupus anticoagulant test.

14. The method according to claim 13, wherein said lupus anticoagulant (LA) test comprises dilute Russell's viper venom time (dRVVT) or LA-responsive activated partial thromboplastin time (aPTT), or a combination thereof.

15. The method according to claim 1, wherein the subject is a patient which has been treated with a direct anticoagulant.

16. The method according to claim 1, wherein the subject is a subject of which the medical history is not known and/or cannot be ascertained.

17. A diagnostic kit for the in vitro diagnosis of a haemostasis disorder comprising
activated charcoal;
a vial comprising a filter, wherein the vial has a volume of between 100 μl and 10000 μl; and
optionally one or more compounds required for the in vitro diagnosis of a haemostasis disorder, optionally wherein the filter has a pore size in a range of 0.22 μm to 0.65 μm.

18. A method for preparing a sample for in vitro diagnosis of a haemostasis disorder comprising the steps of
contacting a volume of between 100 μl and 10000 μl of said plasma sample with activated charcoal in a vial; and
recovering said plasma sample from said activated charcoal by passing said plasma sample through a filter with a pore size in a range of 0.40 μm to 0.65 μm.

19. The method according to claim 1, wherein said plasma sample is contacted with activated charcoal for at least 5 minutes.
